(19) United States
(12) Patent Application Publication
(10) Pub. No.: US 2005/0192484 A1
(43) Pub. Date: Sep. 1, 2005

(54) Retractor blades for minimally invasive surgical procedures and method of retraction

(75) Inventors: Todd William Sharratt, Stillwater, MN (US); Steven M. LeVahn, Lino Lakes, MN (US); Kritaps J. Keggi, Waterbury, CT (US)

Correspondence Address: Z. Peter Sawicki;Westman, Champlin & Kelly, Suite 1600, 900 Second Avenue South, Minneapolis, MN, 55402-3319, US

(73) Assignee: Minnesota Scientific, Inc., St. Paul, MN (US)

(21) Appl. No.: 11/033,982

(22) Filed: Jan. 12, 2005

Related U.S. Application Data
(60) Provisional application No. 60/535,911, filed on Jan. 12, 2004.
(60) Provisional application No. 60/541,625, filed on Feb. 4, 2004.

(51) Int. Cl. A61B 1/32

(52) U.S. Cl. 600/210

(57) ABSTRACT

A retractor for use in a minimally invasive surgical procedure includes a handle having a blade attached to the handle. The blade includes a flesh retaining portion that attaches to and extends from the handle. A flesh engaging portion extends from the flesh retaining portion where a width of the flesh engaging portion is less than or equal to two centimeters. A tab extends from a distal end of the flesh engaging portion and wherein the tab separates flesh from the surgical site.

### CROSS REFERENCE TO RELATED APPLICATION(S)

The present application claims priority of U.S. Provisional Application No. 60/535,911 filed on Jan. 12, 2004, which is hereby incorporated by reference in its entirety.

The present application also claims priority of U.S. Provisional Application No. 60/541,625 filed on Feb. 4, 2004, which is hereby incorporated by reference in its entirety.

### BACKGROUND OF THE INVENTION

The present invention relates to minimally invasive procedures. In particular, the present invention relates to retractor blades that are useful in minimally invasive surgical procedures.

Prior to the introduction and use of endoscopes and fiberoptic lights, surgical procedures were performed by making an incision about a surgical site large enough to expose the tissue, organ, or bone upon which the surgical procedure was performed. However, in some procedures, the damage caused by the incision required excessive rehabilitation and caused more discomfort to the patient than the surgical procedure that was performed.

With the introduction of endoscopes and fibertoptic lights, the surgeon can access portions of the body through a small incision and utilizing minimally invasive surgical techniques. Minimally invasive surgical procedures allow the surgeon to minimize the surgical wound or wounds needed to gain access to the surgical site, whether a tissue, organ, or bone. By minimizing the surgical incision, the recovery time after the surgical procedure has been shortened and minimizing the surgical incision also minimizes the discomfort felt by the patient.

### SUMMARY OF THE INVENTION

The present invention includes a retractor for use in a minimally invasive surgical procedure. The retractor includes a handle having a blade attached thereto. The blade includes a flesh retaining portion that attaches to and extends from the handle. A flesh engaging portion extends from the flesh retaining portion where a width of the flesh engaging portion is less than or equal to two centimeters. A tab extends from a distal end of the flesh engaging portion and wherein the tab separates flesh from the surgical site.

The present invention also includes a kit for performing a joint replacement surgery. The kit includes at least one retractor for retracting flesh to expose the joint for surgery and at least one prosthetic insert for replacing a damaged portion of the joint.

The present invention also includes a method of retracting flesh from a joint that includes incising a surgical wound into the patient proximate the joint and positioning a table mounted retractor support apparatus proximate the surgical wound. A retractor blade having an outwardly extending distal end is inserted into the surgical wound and the distal end is positioned proximate a bone. Manual force is placed upon a handle attached to the retractor blade such that the distal end engages the bone and functions as a fulcrum as the flesh is retracted form the surgical site. With the flesh retracted from the surgical site, the handle is secured to the retractor support apparatus which secures the retractor in a retracting position.

### BRIEF DESCRIPTION OF THE DRAWINGS

FIG. 1 is a perspective view of minimally invasive retractor blades of the present invention being used to retract flesh in a minimally invasive procedure about a hip joint.

FIG. 2 is a top view of the minimally invasive retractors of the present invention being used to retract flesh in a minimally invasive manner about a hip joint.

FIG. 3 is a perspective view of a retractor blade of the present invention.

FIG. 4 is side view of the retractor blade of the present invention.

FIG. 5 is a sectional view along section line 5-5 in FIG. 3 of the retractor blade of the present invention.

FIG. 6 is a perspective view of an alternative embodiment of the retractor blades of the present invention.

FIG. 7 is another perspective view of the alternative embodiment of the retractor blades of the present invention.

FIG. 8 is another alternative embodiment of the retractor blades of the present invention.

FIG. 9 is an additional perspective view of the second alternative embodiment of the retractor blades of the present invention.

FIG. 10 is a sectional view of the retractor of the second alternative embodiment of the present invention retracting the iliopsoas tendon and flesh.

FIG. 11 is a perspective view of retractors having offset tabs from a center of the retractor blade.

FIG. 12 is a perspective view of a retractor blade having an outwardly arcuate distal end of the present invention.

### DETAILED DESCRIPTION OF THE PREFERRED EMBODIMENTS

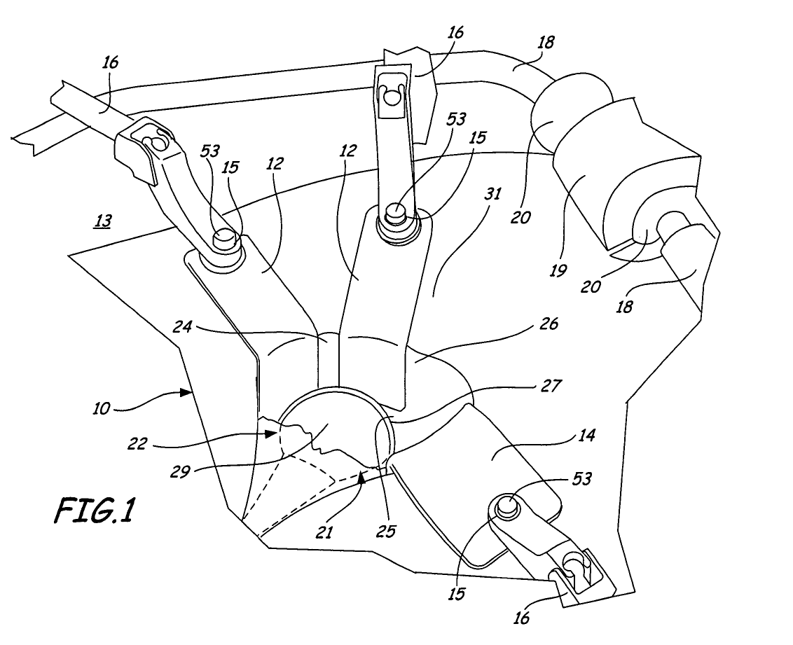

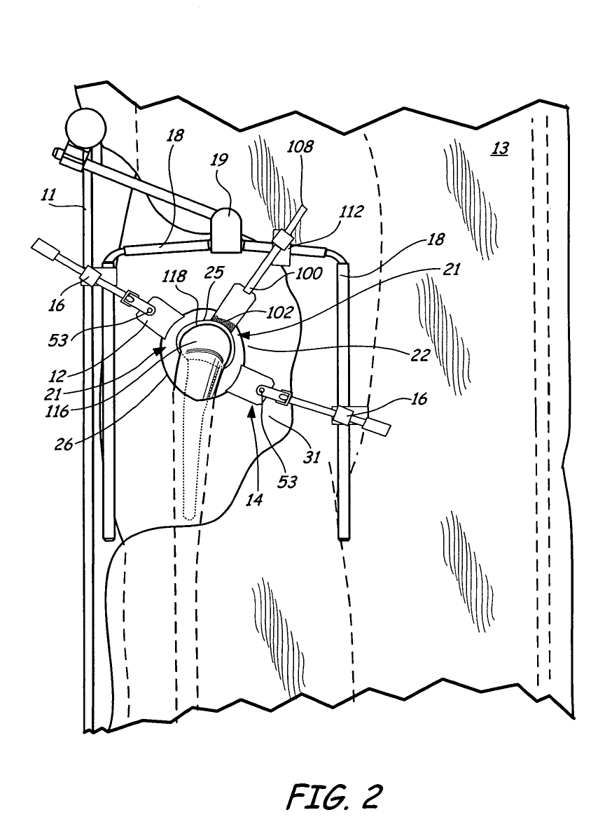

A retractor apparatus for performing a minimally invasive hip surgery is generally illustrated at 10 in FIG. 1. Retractor blades 12 and 14 are designed for minimally invasive surgical procedures are positioned within an incision 26. The retractor blades 12,14 are attached to retractor blade holders 16, which are supported by support arms 18 that are independently movable with respect to each other. Preferably the support arms 18 have pivot balls 20 that are positioned within a clamp 19 that is mounted to a rail 11 of a surgical table 13 as best illustrated in FIG. 2.

The minimally invasive retractor blades 12, 14 are designed to separate and retract flesh 24 to expose a hip joint while minimizing trauma to the flesh 24 that is retracted to gain access to the hip joint 21. By flesh is meant skin, muscle, ligaments, tendons, blood vessels and other body components. Widths of the minimally invasive retractor blades 12, 14 are minimized to minimize a length of the incision 26 as well as the amount of flesh 24. An exemplary range of widths of the minimally invasive retractor blades 12, 14 is between one-half and five centimeters, and preferably between one and two centimeters. The minimized length of the incision 26 and retraction of the flesh 24 reduces trauma, discomfort and recovery time for the patient.

What is meant by minimally invasive is the smallest incision and retraction of flesh 24 around a surgical site 22 that is necessary to perform a surgical procedure. The length of the incision 26 for a minimally invasive hip surgery is about 12 centimeters or less and preferably less than about 8 centimeters. While being described for a surgical procedure on a hip joint 21, the minimally invasive blades 12, 14 can also be used to perform minimally invasive surgical procedures on other areas of the body including a knee joint and a shoulder joint.

The retractor handles 16 provide for horizontal, vertical and lateral adjustment of the minimally invasive retractor blades 12,14 within the surgical site 22. However, the retractor blades 12,14 may be fixedly attached to a rigid handle.

Referring to FIGS. 3-5, the minimally invasive retractor blade 12 includes an arcuate cross-sectional flesh retracting portion 30. A concave arcuate front surface 32 of the flesh retracting portion 30 substantially conforms to an outer surface of a pelvic bone 25 defining an acetabuleum 27 to aid in positioning either blade 12,14 in a selected position.

A convex arcuate back surface 34 of the flesh retracting portion 30 is also designed to conform to a shape of the flesh 24 when retracted from the surgical site 22. The convex back surface 34 minimizes localized pressure points on the flesh 24 and thereby minimizes damage and trauma to the retracted flesh 24. A length of the flesh retracting portion 30 ranges from between less than one inch to greater than six inches and preferably from between two and five inches.

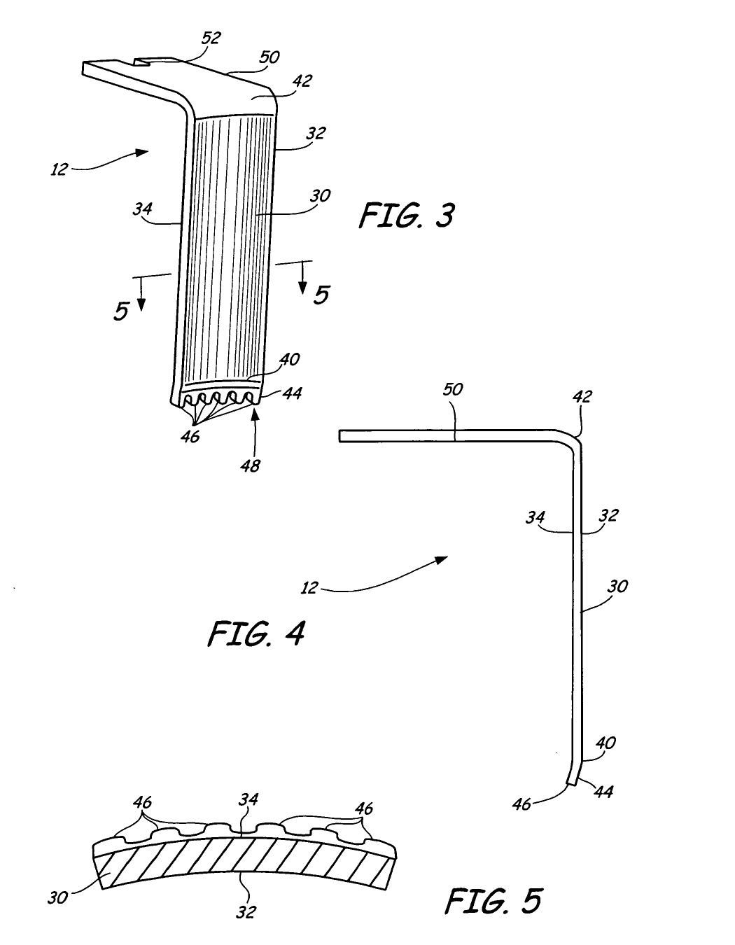

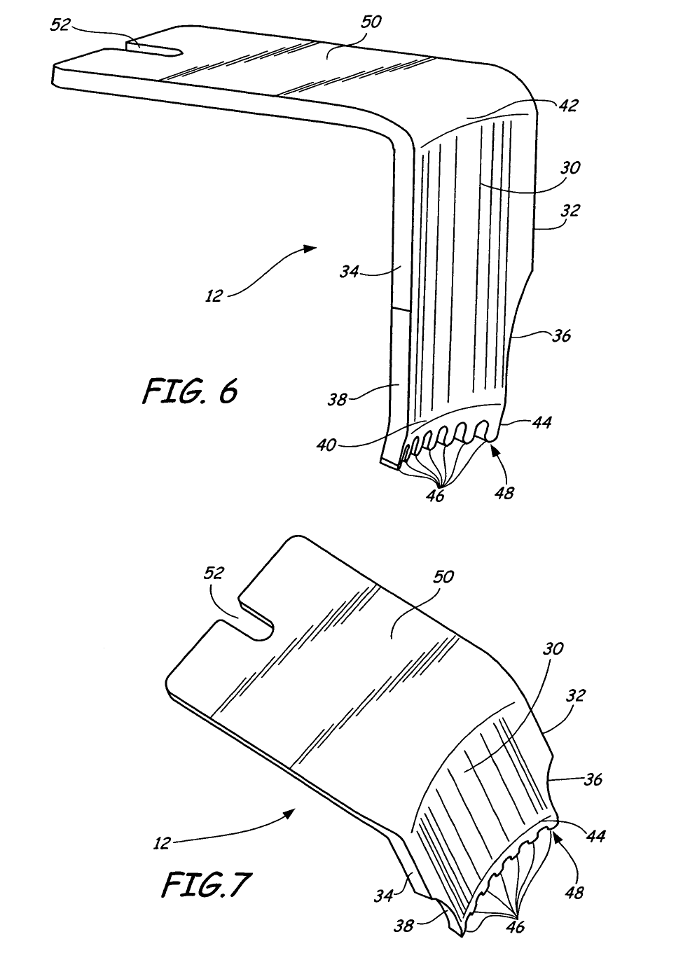

Referring to FIGS. 5 and 6, the blade 12 optionally has arcuate tapered side portions 36, 38 that reduce the width flesh retracting portion 30 at a distal end 40 of the arcuate flesh retracting portion 30. The width of the arcuate flesh retracting portion 30 at the distal end 40 is less than a width of the arcuate flesh retracting portion 30 at a top end 42. The width at distal end 40 is preferably about less than three-fourths of a maximum width of the flesh retracting portion 30, and preferably about less than one-half the width of the maximum width. The reduction in width of the curved flesh retracting portion 30 at the distal end 40 allows either the retractor blade 12,14 to be positioned into confined spaces within the surgical site 22 and also minimizes the amount of the flesh 24 that is retracted about the acetabular rim.

The width of arcuate flesh retracting position 30 ranges between about one-half a centimeter and about three centimeters. Preferably, the width of the flesh retracting portion ranges between about 1 centimeter and about 1.5 centimeters.

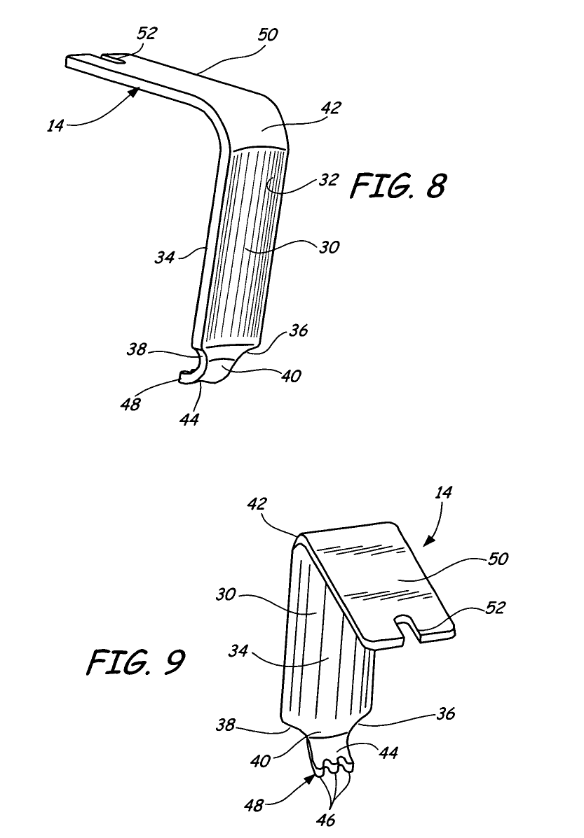

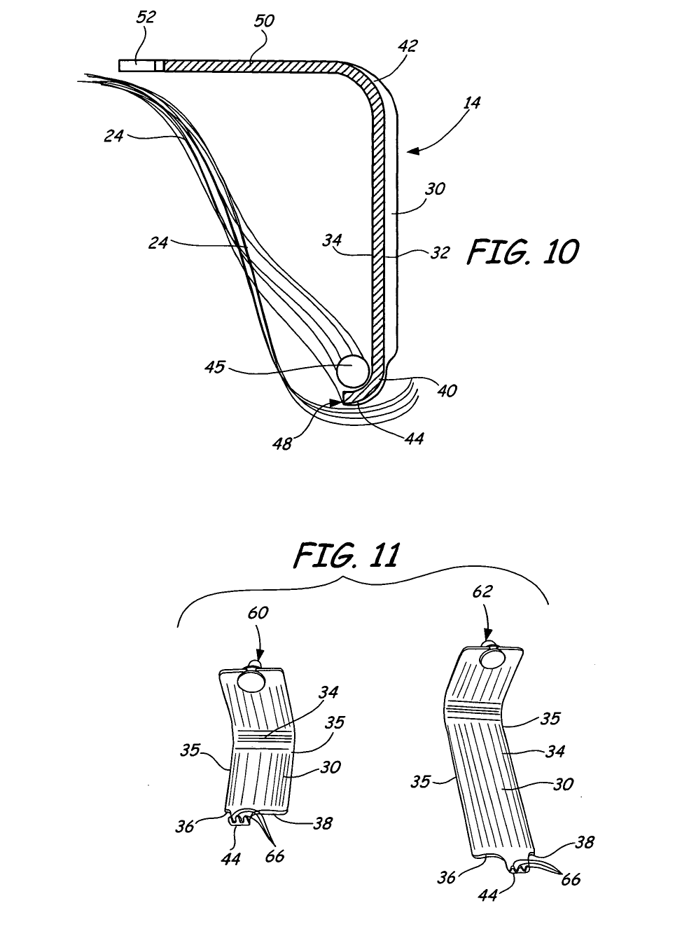

A tab 44 extends from the distal end 40 at a slight angle as illustrated in FIGS. 3-5 to substantially perpendicular from the distal end 40 of the flesh retracting portion 30 as illustrated in FIGS. 8 and 10 on the blade 14. The tab 44 is positioned between the acetabular rim 27 of the pelvic bone 25 and the flesh 24 supporting and surrounding the hip joint 22. Specifically, the tab 44 separates and retracts at least the iliopsoas tendon 45 from the pelvic bone 25 proximate the hip joint 22 without damaging the tendon 45 during the surgical procedure. The tab 44 and the flesh retracting portion 30 engage and retract the iliopsoas tendon 45 as well as flesh 24 as best illustrated in FIG. 10.

The tab 44 is generally centrally located at the distal end 40 of the flesh retracting portion 30 as illustrated in FIGS. 8-10. However, referring to FIG. 11, the tab 44 may be located to a left of center on a blade 60 or to a right of center on a blade 62 of a center of the distal end 36 of the flesh retracting portion 30. Offsetting the tab 40 from the center of the flesh retracting portion 30 allows for retraction of the flesh 24 from confined areas of the hip joint 22 where the selected flesh 24 may be retracted without interference from an edge 35 of the flesh retracting portion 30. The specific retractor blade with the offset tab 44 is selected dependent upon the hip joint 22 that is being surgically repaired.

The tab 44 preferably includes a plurality of teeth 46 extending from an end 48 of the retractor blade 10. The teeth 46 are generally aligned in the same plane as the tab 44 and prevent the flesh 24 from slipping below the end 48 of the retractor blade 12,14 thereby providing better access to the surgical site 22. The teeth 46 also extend into the flesh 24 and prevent the flesh 24 from slipping beyond the teeth 46 and into the surgical site 22.

However, the teeth 46 are not necessary to practice the invention. The tab 44 may have a substantially smooth surface, a knurled surface or any other configuration that allows the minimally invasive surgical procedure to be performed.

A top substantially flat portion 50 of the retractor blade 12, 14 includes a slot 52, which engages a cylindrical peg 53 that extends into the retractor blade holder. One skilled in the art will recognize that the retractor blades 12, 14 can be detached from the retractor blade holder 16 by disengaging the peg 53 from an aperture 15 in the holder 16 as best illustrated in FIGS. 1 and 2. The retractor blade 12, 14 can also be disengaged from the peg 53 by disengaging the top flat portion 50 of the retractor blade 12, 14 from the peg 53. The attachment is disclosed in U.S. Pat. No. 5,882,298, which is hereby incorporated by reference in its entirety. Although the present minimally invasive retractor blades 12, 14 are shown to be removably secure within a retractor blade holder 16, one skilled in the art will recognize that the retractor blades 10 of the present invention can also be fixedly attached to retractor handles while still being within the scope of the present invention.

Referring to FIGS. 3, 4 and 6-11, the top substantially flat portion 50 extends substantially perpendicularly from the flesh retracting portion 30 of the retractor blade 12 and referring to the blade 14 substantially parallel to the tab 44. The top substantially flat portion 50 is substantially longer than a top portion of a typical retractor blade. The top portion 50 is in a range of about one-quarter a length of the flesh retracting portion 30 and about equal to a length of the flesh retracting portion 30. The length of the top portion 50 is designed to engage skin 31 of the patient as the flesh 24 retracted from the surgical site. By engaging the skin 25 of the patient, the substantially top flat portion 48 prevents the flesh 24 from extending upward and about the retractor blade 12,14 which impairs access to the surgical site 22. The length of the top portion 50 also allows the retractor blade holder 16 to be attached a distance from the surgical site 22 and such that the retractor blade holder 16 does not restrict access to the surgical site.

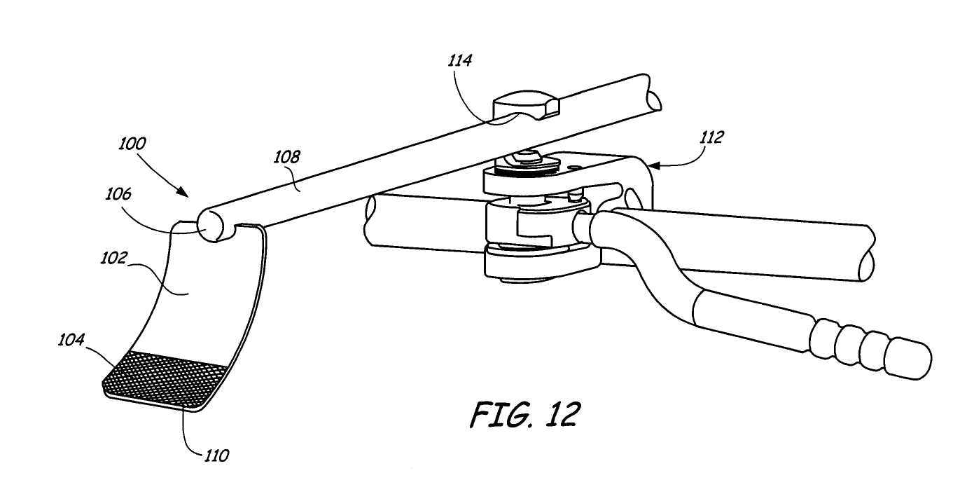

Referring to FIGS. 2 and 12, the flesh 24 may also be retracted with a retractor 100 having a blade 102 having a distal end 104 with an arcuate configuration that extends away from a handle 108 attached to a proximal end 106 of the blade 102. The blade 102 is preferably fixedly attached to the handle 106. However, other attachment mechanisms that attaches to the handle 108 and to the blade 102 are within the scope of the present invention.

The distal end 104 of the blade 102 is manually positioned within the surgical site 22 proximate a rigid anatomical structure such as the pelvic bone 25 proximate the acetabulum 27. Manual force is placed downwardly upon the retractor handle 108 such that the distal end 104 engages the pelvic bones and functions as a fulcrum to retract the flesh 24 from the surgical site 22.

Preferably, the distal end 104 has a rough knurled surface 110 to prevent slippage as the flesh 24 is retracted. However a smooth surface at the distal end is also within the scope of the present invention.

The retractor 100 is retained in the retracting position by attaching the retractor handle 108 to a clamp 112 attached to the retractor support arm 18. Preferably, the clamp 112 has a clamping socket 114 for accepting the handle 108. By socket is meant an opening or a cavity into which an inserted part, such as a retractor support apparatus, is designed to fit and wherein the retractor support apparatus can be inserted into the socket from an infinite number of directions in a 180° range starting from a substantially parallel position to a back surface of the socket to a position substantially perpendicular to the back surface and continuing to position again substantially parallel to the back surface of the socket. However, a clamp 112 with a clamping bore is also within the scope of the present invention.

With the handle 108 positioned with the clamping socket 114 the clamp 112 is positioned into the clamping position and thereby secures the retractor 100 in the retracting position.

Referring to FIGS. 1 and 2, with the hip joint 21 exposed for the surgical procedure such as a total hip replacement, the femoral ball 29 is replaced with a prosthetic ball 116 by any of a number of known methods. The acetabulum is also reamed to accept an insert 118. The prosthetic ball 116 is reducted into the insert 118 to complete the total hip replacement surgery. The retractor blades 12, 14 are removed and the incision 46 is sutured shut. Besides a total hip replacement surgery, other surgical procedures can be performed using minimally invasive retractor blades 12,14 and methods of retraction such as a partial hip replacement surgery, a total or partial knee replacement surgery and a total or partial shoulder replacement surgery.

Although the present invention has been described with reference to preferred embodiments, workers skilled in the art will recognize that changes may be made in form and detail without departing from the spirit and scope of the invention.

1. A retractor for use in a minimally invasive surgical procedure, the retractor comprising:
a handle;
a flesh retaining portion attached to the handle and extending therefrom;
a flesh engaging portion extending substantially perpendicularly from the flesh retaining portion and wherein a width of the flesh engaging portion is less than or equal to two centimeters; and
a tab extending from a distal end of the flesh engaging portion and wherein the tab separates flesh from the surgical site.

2. The retractor of claim 1 and wherein the flesh retracting portion comprises a substantially arcuate cross-section having a substantially convex first surface and a substantially concave second surface.

3. The retractor of claim 1 and wherein a width of the flesh retracting portion decrease at a distal end to provide for retraction in confined spaces.

4. The retractor of claim 1 and wherein the tab is substantially centrally located at the distal end of the flesh retracting portion.

5. The retractor of claim 1 and wherein the flesh separating portion is offset toward a left side or a right side of the flesh retracting portion.

6. The retractor of claim 1 and wherein the tab comprises a plurality of teeth extending from an end thereof.

7. The retractor of claim 1 and the tab extends from the distal end of the flesh engaging portion in a direction substantially parallel to the flesh retaining portion.

8. The retractor of claim 1 and wherein a length of the flesh retaining portion is about less than three-fourths of a length of the flesh retracting portion.

9. The retractor of claim 1 and wherein a length of the flesh retaining portion is about less than one third of a length of the flesh retaining portion.

10. The retractor of claim 1 and wherein the handle fixedly attaches to the flesh retaining portion.

11. The retractor of claim 1 and wherein the handle rotatably attaches to the flesh retaining portion.

12. A kit for performing a joint replacement surgery comprising:
at least one retractor to retract flesh to expose the joint for surgery; and
at least on prosthetic insert for replacing a damaged portion of the joint.

13. The kit of claim 12 and wherein the prosthetic insert comprises:
a femoral ball replacement insert; and
an acetebular insert for performing a total hip replacement surgery.

14. The kit of claim 12 and wherein the retractor comprises:
a handle;
a flesh retaining portion attached to the handle and substantially extending therefrom;
a flesh engaging portion extending substantially perpendicularly from the flesh retaining portion and wherein a width of the flesh engaging portion is less than or equal to two centimeters; and
a tab extending from a distal end of the flesh engaging portion wherein the tab separates flesh and tendons from the surgical site.

15. The kit of claim 14 and wherein the flesh retracting portion comprises a substantially arcuate cross-section having a convex first surface and a concave second surface.

16. The kit of claim 14 and wherein a width of the flesh retracting portion decrease at a distal end to provide for retraction in confined spaces.

17. The kit of claim 14 and wherein the tab is substantially centrally located at the distal end of the flesh retracting portion.

18. The kit of claim 14 and wherein the tab is offset toward a left side or a right side of the flesh retracting portion.

19. The kit of claim 14 and wherein a length of the flesh retaining portion is about less than three-fourths of a length of the flesh retracting portion.

20. A method of retracting flesh from a joint comprising:
incising a surgical wound into the patient proximate the joint;
positioning a table mounted retractor support apparatus proximate the surgical wound;
inserting a retractor blade having an arcuate outwardly extending distal end into the surgical wound;
positioning the distal end of the retractor blade proximate a bone;
providing manual force to a handle attached to the retractor blade such that the distal end engages the bone and functions as a fulcrum as flesh is retracted from the surgical site; and
securing the handle to the retractor support apparatus to secure the retractor in a retracting position.

21. The method of claim 20 and wherein the distal end of the retractor blade includes a knurled surface.

22. The method of claim 20 and further comprising securing the retractor handle within a clamping socket of a clamp attached to the table mounted surgical support apparatus.

23. The method of claim 20 and wherein the joint is the hip joint.

24. The method of claim 20 and wherein the bone is the pelvic bone proximate the acetabulum.
